(19) United States
(12) Patent Application Publication
(10) Pub. No.: US 2009/0198269 A1
(43) Pub. Date: Aug. 6, 2009

(54) Device for the Removal of Thrombi From Blood Vessels

(76) Inventors: Ralf Hannes, Dortmund (DE); Manuel Schneider, Hattingen (DE); Holgar Pracht, Herne (DE); Elina Miloslavski, Bochum (DE); Hermann Monstadt, Bochum (DE)

Correspondence Address: BERLINER & ASSOCIATES, 555 WEST FIFTH STREET, 31ST FLOOR, LOS ANGELES, CA, 90013, US

(21) Appl. No.: 12/086,018

(22) PCT Filed: Dec. 11, 2006
(86) PCT No.: PCT/EP2006/011900

§ 371 Date: Oct. 23, 2008

(30) Foreign Application Priority Data
Dec. 12, 2005 (DE) 102005059670.3

(51) Int. Cl. A61F 2/01

(52) U.S. Cl. 606/200

(57) ABSTRACT

The invention relates to a device for the removal of foreign objects and thrombi from body cavities and blood vessels (12) using at least one guide wire (6, 7) provided with a distal element (2), said distal element (2) being provided with fibers (3) projecting radially outward and the device being provided with a cage (1) or tubular structure (16) which is suitable to be flatly collapsible under the external strain exerted by a micro-catheter (13) and transported inside the micro-catheter (13) and unfolds to its full cage (1) or tubular (16) structure when said external strain caused by the micro-catheter (13) is omitted, with said distal element (2) and the cage (1) or tubular (16) structure being designed so as to be longitudinally movable in relation to each other and the cage (1) or tubular structure (16) having an opening at the distal end through which the distal element (2) can be introduced into said cage (1) or tubular (16) structure. With the help of the inventive device thrombi (5) can be safely eliminated from blood vessels (12) in a simple manner.

The invention relates to a device for the removal of foreign bodies and thrombi from body cavities and blood vessels using at least one guide wire provided with a distal element.

Thromboembolic diseases such as cardiac infarction, pulmonary embolism, peripheral thrombosis, organ embolisms etc. are typically caused by a thromboembolism (hereinafter for short thromb or thrombus), i.e. a visco-elastic blood clot comprising platelets, fibrinogen, coagulation factors etc. forming in a blood vessel which it obstructs either wholly or in part. The obstruction of organ. arteries also leads to the supply of oxygen and nutrients to the associated tissue being interrupted. The disorder of the functional metabolism linked with functional losses is closely followed by a failure of the structural metabolism resulting in the relevant tissue becoming destroyed (infarction). Organs most frequently affected in this way are the heart and the brain. Nevertheless, the arteries of the limbs as well as pulmonary arteries are also impaired. Venous thromboses and thromboembolic occlusions are frequently occurring in the leg and pelvic veins. The disease pattern of the thrombotic occlusion of an intracranial sinus may lead to severe intracerebral hemorrhage due to a failure of venous drainage of brain tissue.

In view of the severity of the disease patterns associated with thromboembolism and the prevalence rate of such diseases various techniques have been developed aimed at dissolving or removing thrombi.

It is known in this context to treat such patients with thrombolytic agents such as streptokinase or urokinase or anticoagulants intended to achieve thrombolysis or limit the growth of thrombi. Since treatment methods of this kind are usually very time consuming they are frequently combined with invasions aimed at reducing the size of or removing the thrombus or embolus mechanically.

Aside from open surgical operations prior art techniques more and more embrace the use of transluminal or endovascular, catheter-guided interventional therapy methods because these are of less invasive nature. It is thus known to remove the thrombus from the patient's body by means of vacuum producing suction catheters or mechanically using catheters provided with capturing cages, helixes, hooks or similar elements; refer to U.S. Pat. No. 6,245,089 B1, U.S. Pat. No. 5,171,233 A1, Thomas E. Mayer et al., Stroke 2002 (9), 2232.

Disadvantages associated with the known transluminal devices are that with said devices it is often impossible to remove the thromb completely and, moreover, there is a risk of the thromb or fragments of it being released into the blood stream thus passing on to vessels of smaller lumen which are more difficult to be reached and treated. Furthermore, due to their size and/or low flexibility the devices known from prior art are only inadequately suited for the removal of thrombi from greatly convoluted vessels or those of particularly small lumen such as those in the brain.

From U.S. 2002/0049452 a device with a catheter is known for the removal of thrombi to which distal end capture arms made of shape-memory material are attached which in their compressed state rest against the catheter and when expanded extend radially from the catheter outwards. When in expanded position which is caused by the body temperature the capture arms are intended to get caught in the thrombus and then retract it out of the blood vessel as the catheter is pulled back into another catheter. The drawback associated with this device is, however, that in order to cool and thus keep the capture arms below transformation temperature before they are released into the blood stream it must either be moved past the thrombus in a secondary catheter which brings about the cooling effect, or a heating system has to be arranged inside the catheter provided with the capture arms that enables the transformation temperature to be attained when the thrombus has been reached. Not only are the design requirements of this configuration very high and thus prone to disturbances it is also the sheer physical size of this device that rules out a treatment of vessels having a particularly small lumen.

In view of the disadvantages of these prior art devices it is thus the object of the invention to provide a device for the removal of foreign bodies and thrombi from body cavities and blood vessels which alleviates the surgical risk existing when removing thrombi and allows the treatment of vessels of especially small lumen.

According to the invention this objective is achieved by providing a device for the removal of foreign objects and thrombi from body cavities and blood vessels using at least one guide wire provided with a distal element, with said distal element being provided with fibers projecting radially outward and the device being provided with a cage or tubular structure which is suitable to be flatly collapsible under the external strain exerted by a micro-catheter and transported inside the micro-catheter and unfolds to its full cage or tubular structure when said external strain caused by the micro-catheter is omitted, with said distal element and the cage or tubular structure being designed so as to be longitudinally movable in relation to each other and the cage or tubular structure having an opening at the distal end through which the distal element can be introduced into said cage or tubular structure.

The basic principle of the invention involves the provision of a device that primarily is composed of two elements that by interaction enable thrombi to be safely eliminated from blood vessels. One of these elements is the distal element provided with fibers or bristles projecting radially outward whereas the other element constitutes the cage or tubular structure. The guide wire(s) designed so as to serve as insertion aid yield excellent maneuvering characteristics even in small-lumen and convoluted vessel segments. The fibers of the distal element are suited to capture and stabilize a thrombus, especially if they are made of or finished with thrombogeneous materials.

The device is transferred to the application site with the aid of a small-lumen micro-catheter. The device situated inside the micro-catheter may either be 1) maneuvered to the distal location of the thrombus and then retracted, 2) released from the micro-catheter in the area of the thrombus, or 3) pushed out of the micro-catheter at a point proximally to the thrombus and then penetrate the thrombus anterogradely. As long as the distal element is confined within the micro-catheter the flexible fibers are pressed onto the distal element in proximal direction due to the mechanical resistance. When the distal element has left the micro-catheter the fibers are capable of unfolding fully and for the main part protrude radially outward perpendicular to the distal element. For the purpose of removing a thrombus (clot) the procedure usually followed is to transport the distal element contained in the micro-catheter to a point distally of the clot since while being inside the micro-catheter the fibers are prevented from standing upright completely so that the diameter of the distal element in this condition is comparatively small. Distally of the clot the distal element will then be expelled from the micro-catheter so that its complete circumferential size unfolds due the fibers now moving into upright position. As a next step the micro-catheter may now be removed. Following this, the distal element is moved into proximal direction with the fibers taking the clot along. The fibers hook themselves into the clot and may thus also serve clot stabilizing purposes.

On the other hand, the cage or tubular structure that also forms part of the device is positioned proximally of the clot. Being retracted in proximal direction the distal element together with the clot can now be pulled through the distal opening of the cage or tubular structure into said structure, which secures the captured thrombus altogether, especially radially outward but, as the case may be, also in proximal direction. Subsequently, the entire device including cage or tubular structure and distal element is retracted farther until it is finally contained in a catheter which is then removed from the blood vessel system. In this manner, an efficient and safe removal of thrombi especially from small-lumen blood vessels can be realized.

As a rule, the latter catheter is a so-called guide catheter having a greater inner diameter than the micro-catheter used for the placement of the device. In this way, the entire thrombus as well as the device in its expanded state can be moved and placed into the guide catheter. During treatment also the micro-catheter is usually pushed forward through the guide catheter although said guide catheter can only be moved forward up to a certain point because in vessels of particularly small lumen, especially in intracranial areas, only the micro-catheter which has a very small diameter can be employed.

It is understood that for the intended purpose the fibers must have adequate stiffness but at the same time must be flexible or bendable enough so that they can be passed through a catheter and do not damage the vessel walls.

The fibers may consist of a natural substance, polymer material, monomers, metal, ceramic material, glass or a combination thereof. Especially preferred are polymer materials.

Suitable materials are primarily polyurethane, polyacrylics, polyester, polytetrafluoroethylene, polyamide or polyalkylene and, due to its peptide-like bond structure, most notably polyurethane and polyamide, e.g. nylon, which enables the thrombus to excellently anchor or “adhere” to the fibers.

Aside from polymer materials metals also well suited for the intended purpose. Suitable metallic materials for treatment purposes are all metals that do not have detrimental effects on the patients. Especially suited for the described purpose are stainless steel fibers and fibers made of metal alloys having shape-memory properties such as for example nitinol fibers. Fibers made of shape-memory materials offer the advantage that when under the external strain exerted by a micro-catheter they are initially shaped to fit closely and after having been released from the micro-catheter assume a second shape allowing them to freely stick out perpendicularly. Furthermore, gold and platinum are suitable materials as well. Also suited are ceramic materials, fiber glass and carbon fibers.

The cage or tubular structure as well is transported in folded up condition through the micro-catheter. As soon as the external constraint exerted by the micro-catheter is omitted it is capable of unfolding to assume its full, expanded cage or tubular structure. Therefore, the cage or tubular structure preferably consists of a shape-memory material, in particular nitinol, because in this case an automatic or self-acting unfolding of the structure is effected after it has been pushed out of the micro-catheter.

The process of unfolding to the full cage or tubular structure upon omission of the external strain exerted by the micro-catheter must not necessarily take place automatically but may also be effected manually. For this purpose an additional guide wire is conceivable, for example, which upon being moved forward causes the structure to unfold.

Generally, the cage or tubular structure is of oblong, ship-like configuration of a length ranging between 5 and 50 mm with a diameter of between 2 and 6 mm in expanded state. Folding up of the cage or tubular structure in this case under the influence of an external constraint caused by a catheter is normally associated with a stretching of the cage structure. The cage or tubular structure in its entirety should be designed such that both unfolding when being moved out of the micro-catheter as well as collapsing when being retracted into the micro-catheter can take place without difficulty.

In the context of this invention the terms “distal” and “proximal” are to be understood as viewed from the direction of the attending physician. The distal end is thus the end situated away from the attending physician which relates to the components of the device advanced farther into the blood vessel system whereas proximal means facing towards the attending physician, i.e. the proximally arranged components of the device are introduced less far into the blood vessel.

If the phrase ‘longitudinal direction’ is used in this document it is to be understood as denoting the direction into which the device is advanced, i.e. the longitudinal axis of the device also coincides with the longitudinal axis of the blood vessel along which the device is moved forward.

To enable the cage or tubular structure on the one hand and the distal element on the other to be longitudinally movable in relation to each other, it is expedient to provide them with separate guide wires. In this manner the cage/tubular structure and the distal element can be moved independently of one another both in proximal and also distal direction. Especially, the distal element can be maneuvered to a point distally of the clot and then retracted proximally into the cage/tubular structure. Moreover, using separate guide wires makes it possible to easily advance the distal element farther into distal direction than the cage/tubular structure.

In accordance with an alternative embodiment the device may as well be provided with only a single guide wire to which distal end the distal element is attached, with the cage or tubular structure being arranged on the guide wire so as to be movable longitudinally. Important in this respect is that also with a single guide wire the longitudinal movability of the two main components of the device is ensured.

To have adequate control over the cage/tubular structure in the event of the embodiment provided with a single guide wire it is considered expedient to arrange two stops on the guide wire between which the cage/tubular structure can be moved longitudinally along the guide wire. In this case the stop element located farther proximally, which may, for example, be welded onto the guide wire, abuts against the cage/tubular structure when being moved forward so that said structure is carried along in distal direction. When the guide wire is retracted, however, the farther distally located stop element abuts on the cage/tubular structure causing it to be carried along in proximal direction. While with the embodiment provided with only a single guide wire there is less freedom of placement of the cage/tubular structure and distal element, it still offers other advantages, however, in that it is of simpler design.

To make sure the cage/tubular structure is moved along by action of the stop elements on the guide wire, it may, at least at one point, preferably at the proximal end, narrow to such an extent that the inner diameter of the cage/tubular structure at this point is smaller than the outer diameter of the stop elements arranged on the guide wire. For example, the cage/tubular structure may converge in a sleeve-like object which is hollow inside so that the guide wire is capable of passing through the sleeve-like object, while the diameter of the two stop elements, however, is too large for them to pass through the sleeve-like object. In this way, the cage/tubular structure is freely movable on the guide wire between the two stop elements but not beyond said elements.

Since the cage/tubular structure serves to secure the thrombus drawn into it together with the distal element, said structure may be provided with a polymer skin arranged on its radial outer side. Such a polymer skin ensures that no fragments of the clot are permitted to escape to the outside and, moreover, makes sure the clot is protected against influences exerted by the inner wall of the vessels.

Such a polymer material may preferably be polyurethane, but other polymers, such as, for example, PTFE (polytetrafluoroethylene), may also be used for manufacturing purposes.

Alternatively or additionally to a polymer skin the cage structure may also be provided with a fiber or wire braiding or mesh on its radial outer side. Such a braiding or mesh should be dense enough to enable the clot mass to be retained is without difficulty. In case a wire mesh is used, it is thought expedient to also use a material having shape-memory properties, in particular nitinol, for its manufacture.

At its proximal end the cage structure may be closed. In this manner, the clot mass can also be safely secured in proximal direction. However, a cage structure closed off at the proximal end is not absolutely necessary; in fact, also a cage or tubular structure may be used that provides for clot securing in radial direction only because by action of the fiber-covered distal element the clot is retained in longitudinal direction anyway. In this case the configuration can rather be viewed as a tubular than a cage structure.

The cage structure may be composed of three or more, in particular four to six braces extending in longitudinal direction. It is to be noted in this respect that by ‘braces extending in longitudinal direction’ not only braces are meant that are arranged exactly parallelly to the longitudinal axis but also those extending at a certain degree of <90° to the longitudinal axis in distal or proximal direction. As mentioned above with respect to the cage structure the braces preferably consist of a material having shape-memory characteristics.

The braces must be designed so as to be collapsible when the system is retracted into the micro-catheter. Moreover, the braces also serve to enable a polymer skin, if applicable, to spread out. The braces may also serve as a basic supporting structure for a fiber or wire mesh arranged on the radial outer side of the cage structure. If thought expedient, further wires, preferably nitinol wires, may be mounted between the braces, said wires serving as limiting elements and support for the polymer skin or fiber/wire mesh.

The braces of the cage structure may also consist of a wire arranged in the form of loops. Such a wire may extend from proximal to the distal end where it forms a loop, extends back in proximal direction and, having formed into another loop runs back in distal direction. If required, additional loops may be provided in the wire configuration such that the total number of braces is increased in this way. If necessary, cross braces may be arranged between these braces. Since the braces in this case are composed of wires configured such that they form into one or several loops, the number of free wire ends is kept small which also reduces the risk of vessel wall injuries. Otherwise, the wire ends would need to be rounded, possibly, or provided with rounded terminating elements.

In accordance with another embodiment the braces starting out from the proximal end of the cage structure extend radially outward, continue in longitudinal direction distally and, having formed into a loop, partially run back in proximal direction along the circumference of the cage structure. In this case as well the braces may serve as securing points for polymer skin or a fiber/wire mesh or braiding. Furthermore, due to the fact that the braces at the distal end of the cage structure form into a loop a rounding is provided in this location and minimizes the risk of injuries to the wall of the vessel. In the distal area the individual braces may be connected with each other by (laser) spot welds and/or by spiral sleeves. Spiral sleeves of this type may have an oval cross section because two braces are accommodated within this cross section, said braces being interconnected by the spiral sleeve.

As per an alternative embodiment the cage/tubular structure is designed in the form of a tubular structure composed of a rolled metal sheet. In this case the structure is not closed at the proximal end. Moreover, on account of the closed surface of such a tubular structure made of a rolled-in metal sheet no polymer skin or braided structure is required on the radial outer side because the clot is secured by the metal sheet itself. Preferably, the sheet in this case as well consists of a material having shape-memory properties, in particular nitinol, so that a self-expanding tube is provided which increases its diameter automatically upon being pushed out of a micro-catheter. To enable such an expansion to take effect the tube, preferably, is not closed radially but the edges extending in longitudinal direction overlap to a certain extent. The tubular structure after expansion should as a maximum have a diameter preventing the lateral slot from being open, with the edges extending in longitudinal direction abutting at the most. In this way, it can be ensured that the captured clot is secured and is held over the entire circumference. Such a tubular structure may of course be used both in the framework of a device provided with two guide wires and in the framework of a device with a single guide wire only.

To enable the cage/tubular structure to be inserted in and removed from the micro-catheter without difficulty it is considered expedient to design said structure such that it terminates in a single point at its proximal end. In case of a cage structure formed by braces this is ensured in particular by arranging for the proximal ends of the braces to converge centrally and be connected with each other. For example, the braces may terminate in a common sleeve. In the event of an embodiment of the device with a single guide wire only such a sleeve may be hollow inside so that it can be moved on the guide wire in longitudinal direction between two stop elements. In case of an embodiment comprising two guide wires it is expedient, however, to connect such a sleeve directly With one of the guide wires.

In case of a tubular structure consisting of a rolled-in metal sheet rolling of the sheet may be brought about in that the longitudinal edges of the sheet each overlap proximally and distally on one side so that in this case as well the tubular structure converges in one point at least proximally. The metal sheet in this case is rolled slightly diagonally. However, such an embodiment differs from the one described above in that the point on which the structure converges at the proximal end is located on the radial circumference of the tubular structure and not in the center of it.

A structure tapering at the proximal end and converging at a connection point is also viewed expedient because if wrongly positioned the cage or tubular structure can be retracted into the catheter without problems so that it may again be discharged after the catheter has been repositioned. As a result of its tapering structure the cage/tubular structure entering the micro-catheter curls up more closely and again assumes its volume-reduced form with the pull force applied to the guide wire and the forces exerted via the catheter rim interacting.

As per an alternative embodiment the invention provides a device for the removal of foreign objects and thrombi from body cavities and blood vessels using at least one guide wire provided with a distal element, said distal element being provided with fibers projecting radially outward and the device being provided with a wire structure which is suitable to be flatly collapsible under the external strain exerted by a micro-catheter and transported inside the micro-catheter and unfolds to its full wire structure when the external constraint exerted by the micro-catheter is omitted, with the wire structure extending partially or wholly over the distal element.

In accordance with this alternative embodiment of the invention the self-expanding wire structure is permanently connected with the guide wire and extends over the brush-like distal element. Contrary to the embodiments described hereinbefore micro-brush and wire structure cannot be moved longitudinally in relation to each other. Generally speaking, a very short structural shape is achieved in this manner which offers benefits in terms of maneuvering, especially if narrow blood vessels are involved.

When the device is used, i.e. retracted in proximal direction, the wire structure separates the thrombus to be removed from the inner wall of the vessel and transports it in proximal direction. The distal element having a micro-brush shape serves the purpose of moving thrombus and thrombus fragments in proximal direction as well. Due to the self-expansion of the wire structure the micro-brush is centered, especially in the proximal area, and is thus capable of performing its cleaning and filtering function particularly well.

The wire structure preferably consists of a wire braiding or wires extending in a loop-shaped fashion. Such a braiding offers a particular advantage in that it retains its form even in extremely winding vessels and is not subject to oval deformation. This configuration ensures that a permanent contact with the inner vessel walls is achieved. To make sure self-expansion of the wire structure commences upon discharge from the micro-catheter it is considered expedient to use a wire structure made, at least to some extent, of a shape-memory material, in particular nitinol.

Proximal to the distal element the wire structure expediently terminates at a point on the guide wire where it can be secured by means of a suitable sleeve or micro-coil. Usually, the wire structure is open at its distal end, however. The wires may extend particularly from proximal to the distal end of the wire structure where they form into loops and then extend back in proximal direction so that there are no free wire ends within the wire structure itself.

The distal element with its radially projecting fibers is preferably a tapered micro-brush, i.e. the diameter of the distal element increases from proximal to distal corresponding to the increase of the length of the fibers. Experience has shown that such a tapered micro-brush is especially efficient for the elimination of thrombi and thrombus fragments.

In the context of the alternative embodiment just described it shall, furthermore, be noted here that also with respect to the materials used for fibers and guide wire, the design of the wire and cage structure, the connection of the wires with each other and with the guide wire etc. the same applies as has been stated for the embodiments previously described.

Particularly suitable for the treatment of vessels of especially small lumen are fibers having a length of 0.5 to 6 mm and preferably 1.2 to 3 mm so that an outer diameter of 1 to maximum 12 mm of the fiber-carrying part of the distal element is attained even when the fibers are arranged radially. For a particularly atraumatic treatment such outer diameter may be slightly smaller than the inner diameter of the relevant blood vessel.

Expediently, the fibers extend over a distal element length ranging between 0.5 and 5 mm. To make sure the thrombus is sufficiently anchored it is expedient if the fibers are arranged on the distal element of the guide wire with a density ranging between 20 and 100 per cm.

Expediently, the guide wire is made of a medical stainless steel or shape-memory material, preferably nitinol. It is expedient in this case to provide a guide wire/guide wires having an outer diameter ranging between 0.2 and 0.4, preferably between 0.22 and 0.27 mm. A typical guide wire length ranges between 50 and 180 cm.

In accordance with another advantageous design of the device the fibers are arranged spirally along the longitudinal axis of the distal element. This embodiment is especially suited for “piercing” or penetrating into the thrombus as the fiber-carrying portion of the distal element works in the same way as a corkscrew if appropriately manipulated by the attending physician.

In conformity with another advantageous embodiment of the invention the distal element with its radially projecting fibers has a tapered structure after it has been discharged from the micro-catheter, i.e. the radial extension of the fibers which in fact corresponds to the diameter of the distal element increases from proximal to distal. The main advantage of such a tapered “brush form” is that irrespective of the width of the blood vessel to be cleaned at a time there are always at least some portions the fibers of which are of optimum length. Fibers have an optimum length for a given blood vessel especially if the fibers contact the walls of the blood vessel in such a way that they are not bent in distal direction when the device is moved proximally. In this case the cleaning efficiency of the fibers is particularly good. Longer fibers, on the other hand, are bent distally during the return movement in proximal direction so that their cleaning efficiency diminishes whereas short fibers may not even reach the inner wall of the vessel and are thus incapable of providing a cleaning effect at that location anyway.

Additionally or alternatively the fibers in the proximal area of the distal element may also be designed to be harder than in the distal area. The harder fibers in the proximal area in this case mainly serve to scrape off a thrombus adhering to the vessel wall while the softer fibers in the distal area primarily serve to secure or retain the thrombus or fragments of a thrombus.

The fibers to be used according to the invention preferably project at an angle ranging between 70° and 110°, preferably at an angle of between 80° and 90° from the longitudinal axis of the device. These angle indications are to be understood such that angles <90° characterize a proximal orientation of the fibers whereas angles >90° signify a distal orientation of the fibers. Embodiments providing for an angle which is slightly smaller than 90° are particularly atraumatic when upon forward movement in the vessel or through the thrombus and at the same time result in an especially effective anchoring within the thrombus when being retracted.

The fibers may be attached to the distal element by braiding, clamping, bonding, knotting, welding and/or fusing. Techniques which are aimed at connecting fibers in this manner are, for example, known from the fabrication of fiber-equipped embolization spirals.

In accordance with an especially preferred embodiment the distal element is manufactured in such a way that the fibers are placed adjacent to each other and, if so desired, additionally superimposed on each other between two core wires, with said fibers extending orthogonally to the core wires. It is to be noted in this context that according to the invention an orthogonal extension shall not exclusively mean an angle of exactly 90° but rather any transverse configuration of the fibers in relation to the core wires, i.e. the fibers primarily extend transversely to the core wires, not in parallel. Accordingly, also angles of for instance 70° may be viewed as being orthogonal in the framework of the invention. After the fibers have been placed between the core wires, said wires are twisted together, for example in that one end is fixed while the other one is turned or twisted around the other to bring about a plastic deformation of the core wires thus forming into a spiral structure. After the core wires have been twisted together the fibers project outwardly from the twisted core wires virtually in the form of a helical line. The significant advantage of such a distal element is that relatively little core wire is required to achieve a very high fiber coverage. The use of core wires also offers benefits in that the system retains high flexibility. Moreover, fixing the fibers at the core wires in this embodiment is particularly simple and results in the fibers to be distributed in a particularly uniform manner.

Quantity as well as density of the fibers can be controlled, inter alia, via the number of core wire twistings or windings so that different hardness characteristics can be produced with respect to the radial force exerted by the brush-shaped distal element because the higher the number of windings the more fibers can be arranged per unit of length. Moreover, the bending stiffness can be adjusted, inter alia, via the number of core wires and twistings provided.

If thought expedient, the devices may be provided with several distal elements from which fibers stick out radially. Such a system may, for example, offer benefits if particularly large thrombi or, as the case may be, several thrombi have to be eliminated from the blood vessel system. Furthermore, a fiber-covered element located farther distally may serve, if need be, to intercept and remove fragments of a thrombus that detach from and fall off the distal element situated farther proximally.

To enable an adequate flexibility to be achieved despite the length of such a system it is considered expedient to interconnect the individual distal elements by means of connecting elements, especially articulated joints. Such an articulated joint makes it possible for the device to perform within certain limits bending movements and thus follow the configuration of the blood vessels.

The fiber ends located radially outward beneficially are provided with slubs or thicker nubs, for example of spherical shape, so that increased surface is available for better clot mass retention. Another advantage of this embodiment makes it possible in this way to provide fiber ends that have an atraumatic effect. The thicker nubs at the ends of the fibers may for example be produced by cutting the fibers with the aid of methods like micro-laser cutting, electron beam cutting etc.

In accordance with another embodiment the fiber ends located radially outward are at least partially connected with each other via loops. The fibers connected in this manner thus comprise or are made up by a single fiber and not two fibers with the single fiber having a loop-shaped configuration. The fiber projects radially outward, extends up to the outer limit of the expanded distal element, forms a loop and runs back to the center of the distal element. The fibers in this case extend such that an elliptical shape is formed. This embodiment offers advantages in that, similar to the thicker nubs at the end of the fiber, there is a larger surface available for clot mass retention Which results in the thrombus capturing effect being improved. Furthermore, the roundness of the loop makes it atraumatic. Also beneficial is that the fibers have increased stiffness due to the loop-shaped fiber configuration exhibiting a behavior similar to that of two fibers arranged side by side.

It may also be advantageous as the case may be if the fibers protrude, at least partially, differently far radially outward at the sides of the distal element. Similar to the embodiment described hereinbefore in which the radial expansion of the distal element increases from proximal to distal it is achieved in this manner as well that there are always at least some fibers available that are of optimum length to yield the respective cleaning effect. Inter alia, this may be brought about by arranging for the wire(s) from which the fibers of the distal element emanate to extend outside of the center, i.e. extend eccentrically. In this way, relatively short fibers are located on one side whereas relatively long fibers exist on the other side. As a result of their short distance to the point of attachment the short ends of the fibers have significantly harder characteristics and the long ends are significantly softer so that in this case as well the harder fibers rather improve the cleaning effect whereas the softer fibers enable a better retention of the captured thrombus. A distal element with eccentrically arranged wire configuration may offer still another advantage in that such a distal element can be maneuvered past the side of a clot more easily and then accommodate it when retraction in proximal direction takes place.

As per a particularly preferred embodiment of the device the fibers are coated. For example, this coating may be a neutral one consisting of Parylene or polytetrafluoroethylene (Teflon), but may also be comprised of collagen or may be a coating of a material conducive to blood coagulation, preferably having one or several coagulation factors. This embodiment serves to strengthen the anchorage of the fibers inside the thrombus and alleviates the risk of the thrombus disintegrating to such an extent that fragments of it remain in the blood vessel or may be allowed to be released in the blood stream.

Surprisingly, it has been found that a thrombogeneous finishing of the fibers resulted in a significant stabilization of the thrombus at the device provided according to the invention. In this context it is left to the surgeon to bring the inventive device into contact with the thrombus and maintain this contact for a certain period of time thus allowing the thrombogeneous elements to promote an “adherence” of the thrombus to the device. Such an adherence to thrombogeneous fibers is achieved after a relatively short period, even within a few minutes at times. Not only does this preclude the disintegration of the thrombus as it is encountered with many devices, also the retraction of the thrombus into the catheter and its extraction from the vascular system is facilitated in this manner. Especially suited thrombogeneous materials and coatings for this purpose are known from literature to those skilled in the art. Especially suitable to this end are, for example, one or several of the factors fibrin, thrombin, factor XIII and/or factor VIII.

It is, furthermore, advantageous if the fiber-covered distal element is a little longer than the cage/tubular structure. It is ensured in this manner that after the distal element has been drawn into the cage/tubular structure detached thrombus fragments are intercepted within the distal zones of the distal element. The cage/tubular structure is quasi closed off at the distal end by action of the projecting fibers of the distal element. Especially, if the guide catheter has a comparatively small inner diameter in relation to the outer diameter of the device it can be prevented in this manner that the clot mass is squeezed out when the guide catheter is being retracted.

Advantageously, the device is provided with one or several radiopaque markers. These may, for example, consist of platinum or a platinum alloy. Radiopaque markers of this kind may be located both in the area of the distal element and in the area of the cage/tubular structure so that the attending physician will be able to monitor the positioning relative to each other and thus the treatment progress with the help of image-forming methods conducive to the purpose.

Moreover, it is considered advantageous if the tip of the entire device is designed so as to be atraumatic, i.e. is rounded off for example.

Eventually, the invention also relates to the combination of the device with a guide and/or micro-catheter in which the device is maneuvered to the application site and when filled with the thrombus removed from the blood vessel system. It may be expedient to additionally design the catheter in the form of an aspiration catheter capable of accommodating micro-catheters.

The above described invention is of special significance to the removal of thrombi from vessels of especially small lumen, in particular intracranial vessels. The invention may of course be used also for the elimination of thrombi from other parts of the body, for example the heart, lungs, legs etc. However, the invention may also be used for the removal of other foreign objects from blood is vessels, for example removing embolization spirals and stents.

Further elucidation of the invention is provided through the enclosed figures, where

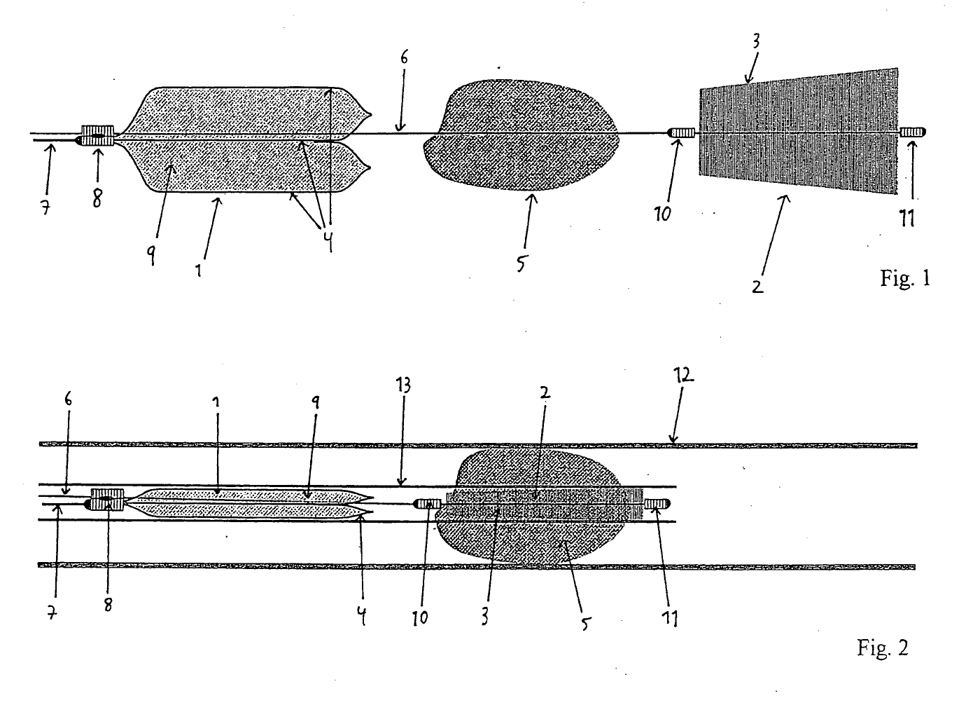

FIG. 1 is a side view showing the inventive device;

FIGS. 2 to 6 is a representation of the inventive device shown in FIG. 1 illustrating various steps of a thrombus removal process;

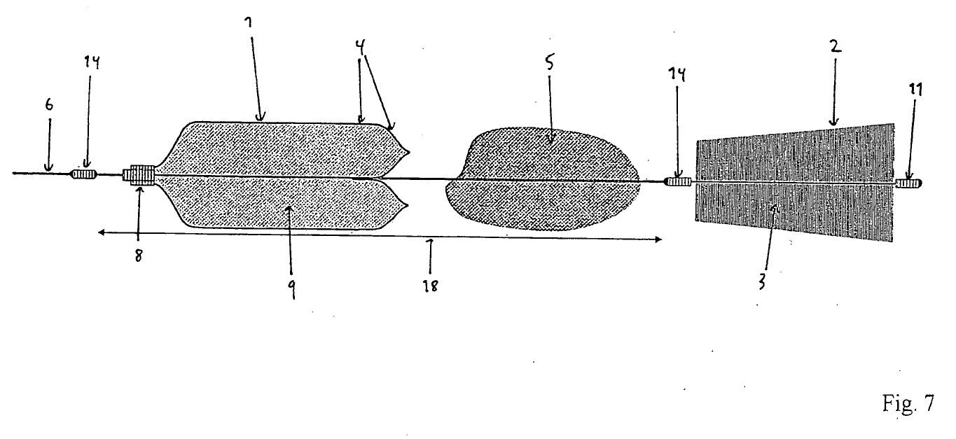

FIG. 7 is a side view of the inventive device in accordance with an alternative embodiment;

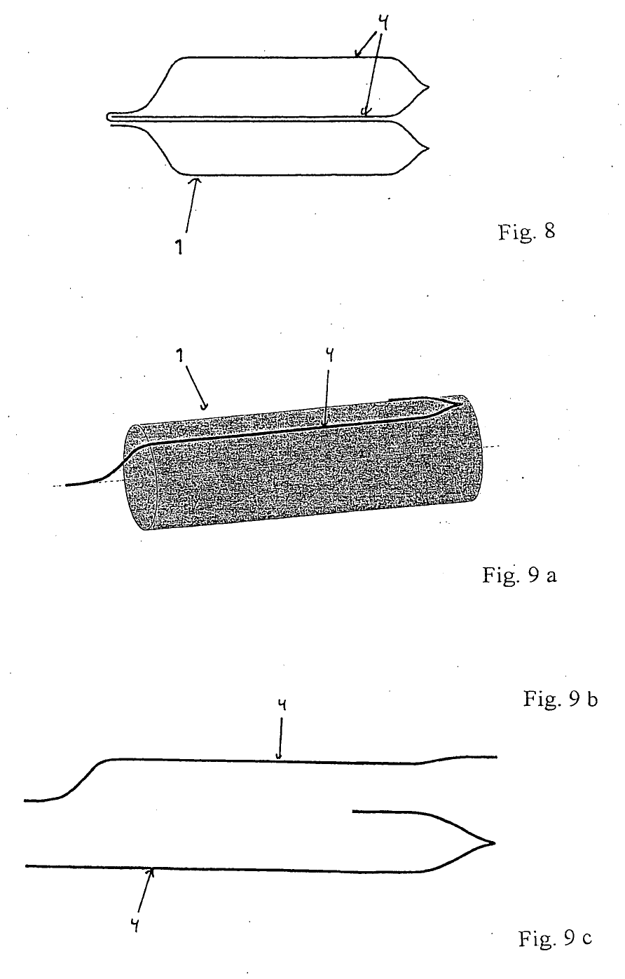

FIG. 8 illustrates the configuration of the braces forming a cage structure;

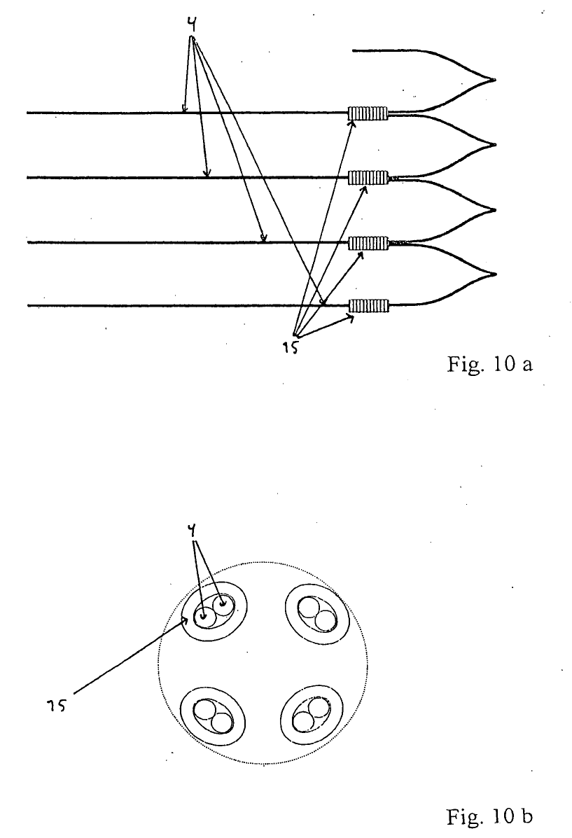

FIGS. 9a, b, c illustrate an alternative configuration of the braces forming a cage structure;

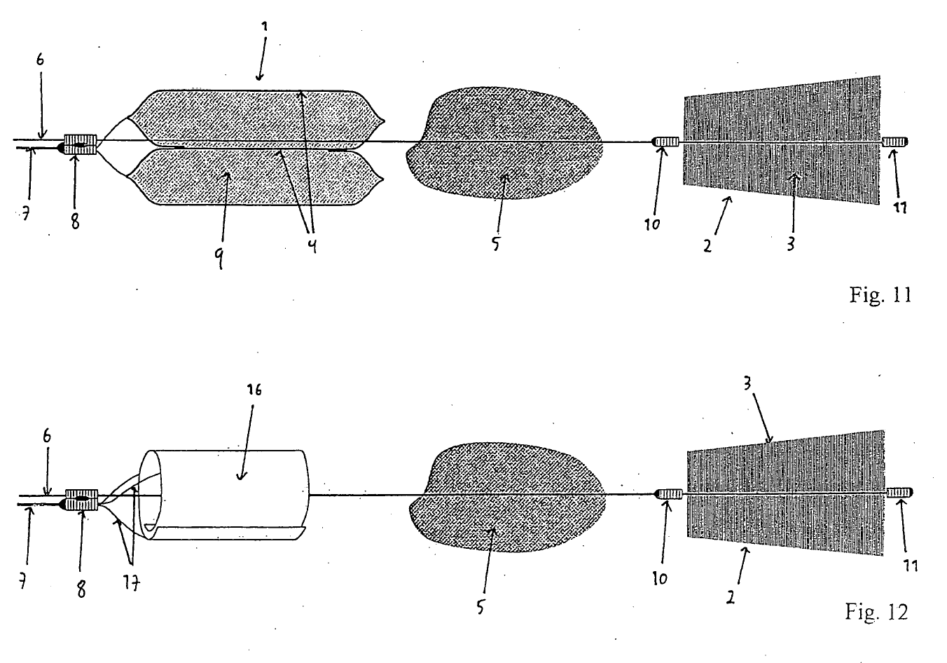

FIG. 10 a shows the connection of braces forming a cage structure in the distal area using spiral sleeves;

FIG. 10b is a cross-sectional view of the cage structure shown in FIG. 10a;

FIG. 11 is a side view of the inventive device in accordance with another embodiment;

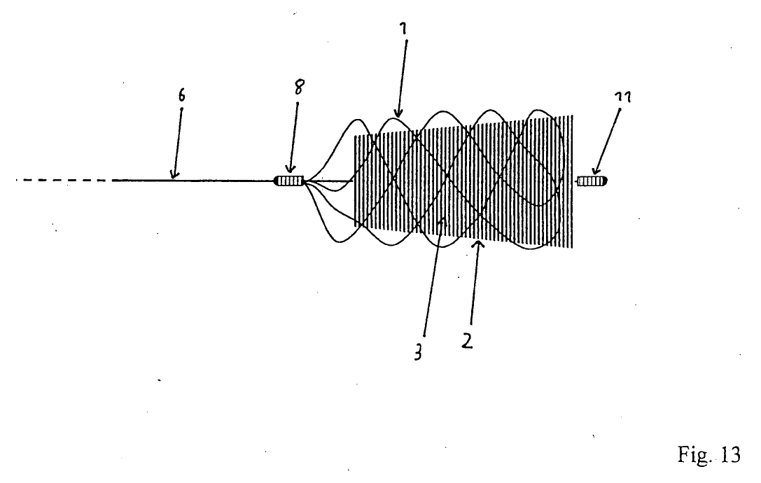

FIG. 12 is a side view of the inventive device showing another embodiment; and

FIG. 13 illustrates an alternative embodiment of the invention.

FIG. 1 is a representation of the first embodiment of the invention shown as a side view. The device in particular is provided with a cage structure 1 as well as a distal element 2 as main components. From distal element 2 fibers 3 project radially outward. The cage structure 1 is composed of braces 4 which for the main part extend in longitudinal direction. The device serves to capture a thrombus 5, initially with the aid of the distal element 2 and fibers 3 projecting from it, by moving the distal element 2 backward in proximal direction and finally maneuvering said thrombus into the cage structure 1. In the side views shown here proximal always denotes to the left, distal to the right. Distal element 2 and cage structure 1 are movable by way of separate guide wires 6 and 7. At its proximal end the cage structure 1 converges centrally in a sleeve 8 to which the guide wire 7 for the cage structure 1 is secured, whereas the guide wire 6 for the distal element 2 extends and passes through the sleeve 8. Continuing, the guide wire 6 extends through the interior of the cage structure 1 so that upon retraction of distal element 2 this slides automatically into cage structure 1 together with the captured thrombus 5. At its distal end the cage structure 1 is open.

Along the radial circumference the cage structure 1 is provided with a polymer skin 9 which serves the purpose additionally securing a captured thrombus. Distal element 2 and guide wire 6 are connected with each other via a micro-coil 10. At the distal end the entire device is provided with a distal tip 11 that is of rounded design and thus has an atraumatic effect. The distal element 2 in its entirety has a tapered structure since the length of fibers 3 increases from proximal to distal. Such an embodiment has the benefit in that irrespective of the width of the blood vessel there are always fibers 3 that are of optimum length. Moreover, during the retraction process the longer fibers 3 arranged in the distal area of the distal element 2 are capable of capturing fragments that may detach from thrombus 5.

FIGS. 2 to 6 illustrate the device shown in FIG. 1 during application. FIG. 2 shows the device situated in a micro-catheter 13 being introduced into a blood vessel 12. In this condition cage structure 1 and also distal element 2 are greatly compressed with the inner diameter of micro-catheter 13 limiting the radial expansion of the device. The micro-catheter 13 is guided laterally past the thrombus 5 or introduced so as to go directly through thrombus 5. In this way the distal end of the micro-catheter 13 is positioned distally to the thrombus 5.

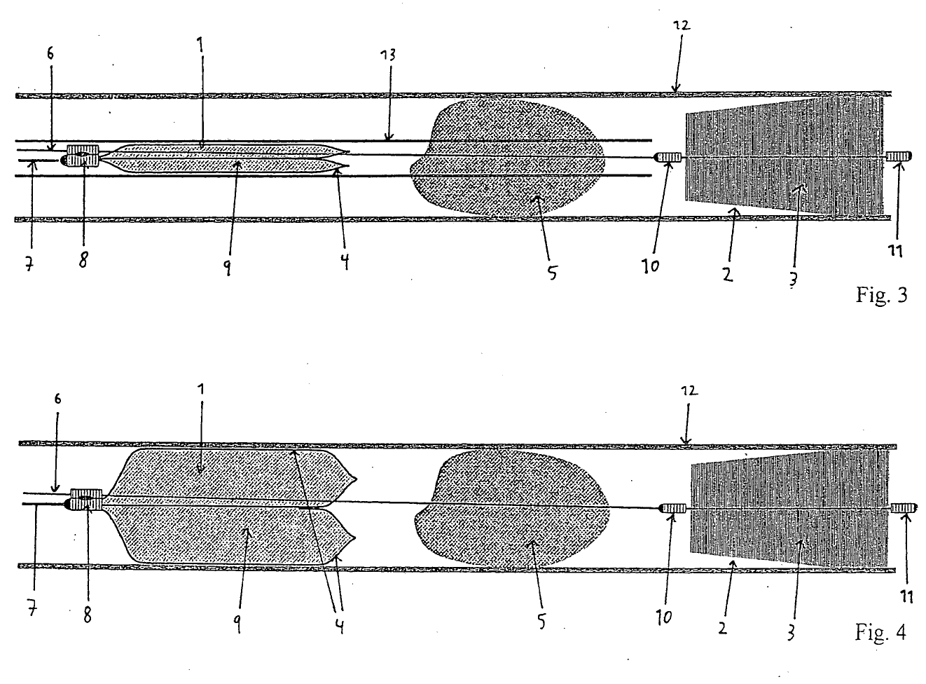

In FIG. 3 the distal element 2 is shown pushed out of micro-catheter 13 and fully unfolded, i.e. the fibers 3 are now projecting radially outward to a much greater extent. On the other hand, micro-catheter 13 still contains the cage structure 1 in compressed condition.

In FIG. 4 the micro-catheter 13 is shown retracted in proximal direction so that also cage structure 1 has now been freed from the micro-catheter 13 and permitted to assume its full cage structure. The outer diameter of the cage structure 1 now coincides roughly to the inner diameter of the blood vessel 12. As is illustrated in FIGS. 2 to 4 it is achieved in this manner that the distal element 2 is positioned distally to thrombus 5 whereas the cage structure I is situated proximal to the thrombus 5.

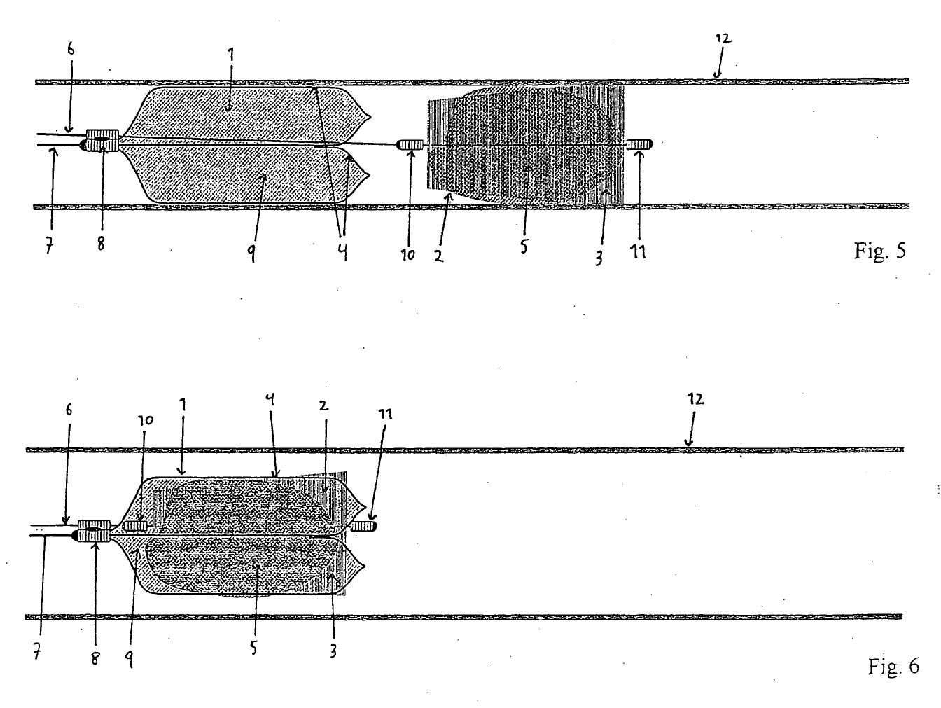

From FIG. 5 it can be seen how the thrombus 5 is captured by retracting the distal element 2 in proximal direction. Fibers 3 now secure and stabilize the thrombus 5 so as to avoid thrombus fragments from becoming split off and going astray in the blood vessel system.

FIG. 6 shows the distal element 2 together with the thrombus 5 being retracted to such an extent that it has entered the cage structure 1. The cage structure 1 must of course be provided with an appropriately large opening at its distal end. The thrombus 5 will now be additionally secured by means of cage structure 1 with polymer skin 9 so that it may be assumed that thrombus 5 is safe and cannot be lost. Subsequently, the entire device is retracted until it is located inside a guide catheter which as an inner diameter of a size adequate to accommodate the entire device. The guide catheter is located in a blood vessel 12 having a larger diameter as can be seen for the blood vessel 12 shown here. Finally, the guide catheter is take out of the blood vessel system as a whole, with the thrombus thus being eliminated entirely.

FIG. 7 shows an alternative embodiment of the invention with only one guide wire 6 provided for the distal element. The cage structure 2 in this case is not provided with a separate guide wire. However, stop elements 14 arranged on the guide wire 6 make sure that the cage structure 1 Which is slidably located in longitudinal direction on guide wire 6 can only be moved between these two stop elements 14. Cage structure 1 can thus only be through the area indicated by arrow 18. The stop elements 14 are designed such that their diameter is too large to enable them to fit through sleeve 8.

When moving the entire system forward through the micro-catheter the cage structure as well is pushed into distal direction due to the effect of the proximal stop element 14. After the distal element 2 has been released at a point distal to thrombus 5 the micro-catheter is retracted in order to liberate also the cage structure 1 proximal to thrombus 5. As a rule said structure maintains its axial position in blood vessel 12 on account of the sufficiently high radial forces it exerts. In the event the cage structure 1 does not maintain its axial position on its own and move in proximal direction the micro-catheter 13 may be used to provide assistance in securing cage structure 1 in that micro-catheter 13 either is retracted just to such an extent initially that the cage structure 1 is permitted to unfold completely or, later, is again moved forward up to the cage structure 1.

Subsequently, as described above, the distal element 2 is retracted with a view to capturing the thrombus 5 until finally the distal element 2 and the thrombus 5 are contained in cage structure 1. When the device is retracted further also the cage structure 1 is moved in proximal direction because the distal stop element 14 carries cage structure 1 along. To complete the process the entire device may be withdrawn in proximal direction into a guide catheter and then removed.

FIG. 8 shows the design of a cage structure 1 comprising braces with said braces 4 in this case consisting of a wire configured so as to form loops. In this manner, only a few wire ends exist that may lead to blood vessel injury. Moreover, due to the bent wire configuration only two distal edges of the cage structure 1 are formed which results in the distal opening of cage structure 1 being sufficiently large. If necessary, additional cross braces may be integrated into the cage structure 1 shown here, especially in the distal area, to even more stabilize the cage structure 1 and give support to the polymer skin 9.

In FIGS. 9a, 9b and 9c a possible configuration of a brace forming a cage structure 1 is illustrated, With said braces starting out from the proximal end of the cage structure 1, extending radially outward, continuing in longitudinal direction distally and having formed a loop on the circumference of the cage structure 1 extending backwards to some extent in proximal direction. FIG. 9b is a side view, FIG. 9c a top view of the brace 4. It can be seen that the offset of the brace 4 in the proximal area is effected so as to be perpendicular to the offset of brace 4 in the distal area.

As is shown in FIG. 10a the distal ends of the braces 4 bent backwards are connected with each other by means of spiral sleeves 15. It is to be observed in this case that FIG. 10a illustrates so to speak an unfolded cage structure 1. In fact, the braces 4 of course extend over the circumference of the cage structure 1.

FIG. 10b is a cross-sectional representation of FIG. 10a from which it can be seen how a total of four spiral sleeves 15 serve to interconnect two braces 4 in each case. Connecting the braces 4 may additionally or alternatively to the spiral sleeves 15 be effected by providing laser spot welds. The number of braces 4 to be provided may, of course, be higher or lower as needed.

In FIG. 11 an alternative embodiment is shown which provides for a cage structure 1 the proximal end of which has not been closed off. Nevertheless, the thrombus 5 can still be adequately secured with the help of the polymer skin 9 covering the radial circumference of the cage structure 1. The shape of the cage structure 1 in this case rather resembles a hose or tube and is not a true cage structure.

FIG. 12 illustrates still another embodiment of the invention which provides for a tubular structure 16 to be used instead of a cage structure, said tubular structure being composed of rolled sheet metal. The lateral ends of the sheet metal in this case overlap to a certain extent, with the diameter of the tubular structure 16, even in expanded condition, not exceeding a diameter that may leave a lateral gap or open lateral slot. At the proximal end the tubular structure 16 is provided with connecting braces 17 serving the purpose of ensuring tubular structure 16 folds up or collapses upon retraction. In the variant shown here the device 2 has separate guide wires 6, 7, but conceivable of course is also an embodiment in which the tubular structure 16 is combined with the aid of only a single guide wire 6.

The alternative embodiment of the invention is illustrated in FIG. 13 and, instead of a cage structure and distal element longitudinally movable in relation to each other, provides for a wire structure 1 extending wholly or partially over the distal element 2. The wire structure 1 is design as a braiding with the individual wires terminating in a sleeve arranged on and permanently attached to the guide wire 6. The wire structure 1 is made of nitinol material and expands in a self-acting manner after it has been pushed out of the micro-catheter. The total number of wires used may vary, the illustration, for example, shows two wires extending spirally from proximal to distal and back such that a mesh or braiding is formed in this manner. However, three or more wires may of course also be employed. The embodiment of the invention shown here is distinguished by its short structural shape which renders it particularly advantageous for application in small, angled vessels.

1. Device for the removal of foreign objects and thrombi (5) from body cavities and blood vessels (12) using at least one guide wire (6, 7) provided with a distal element (2), characterized in that said distal element (2) being provided with fibers (3) projecting radially outward and the device being provided with a cage (1) or tubular structure (16) which is suitable to be flatly collapsible under the external strain exerted by a micro-catheter (13) and transported inside the micro-catheter (13) and unfolds to its full cage (1) or tubular (16) structure when said external strain caused by the micro-catheter (13) is omitted, with said distal element (2) and the cage (1) or tubular (16) structure being designed so as to be longitudinally movable in relation to each other and the cage (1) or tubular structure (16) having an opening at the distal end through which the distal element (2) can be introduced into said cage (1) or tubular (16) structure.

2. Device according to claim 1, characterized in that the fibers (3) consist of a polymer material.

3. Device according to claims 2, characterized in that the fibers (3) are made of polyurethane, polyamide, polyacrylics, polyester, polytetrafluoroethylene or polyalkylene.

4. Device according to any one of the claims 1 to 3, characterized in that the cage (1) or tubular (16) structure consists, at least partially, of a shape-memory material, in particular nitinol.

5. Device according to any one of the claims 1 to 4, characterized in that the cage (1) or tubular (16) structure on the one hand and the distal element (2) on the other are connected by means of separate guide wires (6, 7).

6. Device according any one of the claims 1 to 4, characterized in that the device is provided with only a single guide wire (6) to which distal end the distal element (2) is attached, with the cage (1) or tubular (16) structure being arranged on the guide wire so as to be movable longitudinally.

7. Device according to claim 6, characterized in that the cage (1) or tubular (16) structure is longitudinally movable on the guide wire (6) between two stop elements (14) located on the guide wire (6).

8. Device according to claim 7, characterized in that the cage (1) or tubular (16) structure, at least at one point, preferably at the proximal end of the cage (1) or tubular (16) structure narrow to such an extent that the inner diameter of the cage (1) or tubular (16) structure at this point is smaller than the outer diameter of the stop elements (14) arranged on the guide wire (6).

9. Device according to any one of the claims 1 to 8, characterized in that the cage structure (1) has been provided with a polymer skin (9) on its radial outer side.

10. Device according to claim 9, characterized in that the polymer skin (9) consist of polyurethane.

11. Device according to any one of the claims 1 to 10, characterized in that the cage structure (1) has been provided on its radial outer side with a fiber or wire braiding.

12. Device according to any one of the claims 1 to 11, characterized in that the cage structure (1) is closed at the proximal end.

13. Device according to any one of the claims 1 to 12, characterized in that the cage structure (1) has been provided with three or more, particularly four to six braces (4) extending in longitudinal direction.

14. Device according to claim 13, characterized in that the braces (4) of the cage structure (1) consist of a wire arranged so as to form loops.

15. Device according to claim 13, characterized in that the braces (4) starting out from the proximal end of the cage structure (1) extend radially outward, continue in longitudinal direction distally and, having formed into a loop, partially run back in proximal direction along the circumference of the cage structure (1).

16. Device according to any one of the claims 13 to 15, characterized in that the braces (4) are connected with each other in the distal area by means of laser spot welds and/or spiral sleeves (15).

17. Device according to any one of the claims 1 to 12, characterized in that the tubular structure (16) consists of a rolled-in metal sheet.

18. Device according to any one of the claims 1 to 17, characterized in that the cage (1) or tubular (16) structure at its proximal end terminates in a point.

19. Device for the removal of foreign objects and thrombi (5) from body cavities and blood vessels (12) using at least one guide wire (6) provided with a distal element (2), characterized in that the distal element (2) being provided with fibers (3) projecting radially outward and the device being provided with a wire structure (1) which is suitable to be flatly collapsible under the external strain exerted by a micro-catheter (13) and transported inside the micro-catheter (13) and unfolds to its full wire structure (1) when the external constraint exerted by the micro-catheter (13) is omitted, with the wire structure (1) extending partially or wholly over the distal element (2).

20. Device according to claim 19, characterized in that the wire structure (1) consists of a wire braiding or wires extending in a loop-shaped fashion.

21. Device according to claim 19 or 20, characterized in that the wire structure (1) consists, at least partially, of a shape-memory material, in particular nitinol.

22. Device according to any one of the claims 1 to 21, characterized in that the fibers (3) have a length of between 0.5 and 6 mm and preferably between 1.2 and 3.0 mm.

23. Device according to any one of the claims 1 to 22, characterized in that the fibers (3) are arranged spirally along the longitudinal axis of the distal element (2).

24. Device according to any one of the claims 1 to 23, characterized in that the radial extension of the fibers (3) of the distal element (2) increases from proximal to distal.

25. Device according to any one of the claims 1 to 24, characterized in that the fibers (3) in the proximal area of the distal element (2) are harder than in the distal area of the distal element (2).

26. Device according to any one of the claims 1 to 25, characterized in that the fibers (3) form an angle with longitudinal axis of the device that ranges between 70° and 110°, preferably between 80° and 90°.

27. Device according to any one of the claims 1 to 26, characterized in that the fibers (3) are secured or attached to the distal element (2) by braiding, clamping, bonding, knotting, welding and/or fusing.

28. Device according to any one of the claims 1 to 27, characterized in that the ends of the fibers (3) located radially outward are provided with slubs or nubs.

29. Device according to any one of the claims 1 to 28, characterized in that the ends of the fibers (3) located radially outward are at least in part connected with each other by means of loops.

30. Device according to any one of the claims 1 to 29, characterized in that the fibers (3), at least partially, protrude differently far radially outward at the sides of the distal element (2).

31. Device according to any one of the claims 1 to 30, characterized in that the fibers (3) have been provided with a coating.

32. Device according to any one of the claims 1 to 31, characterized in that the distal element (2) is designed so as to be slightly longer than the cage (1) or tubular (16) structure.

33. Device according to any one of the claims 1 to 32, characterized by one or several radiopaque markers.

34. Device according to any one of the claims 1 to 33 in combination with a guide catheter and/or micro-catheter (13).

35. Device according to claim 34, characterized in that the guide or micro-catheter (13) is designed as aspiration catheter.
